(19) United States
(12) Patent Application Publication
(10) Pub. No.: US 2023/0296589 A1
(43) Pub. Date: Sep. 21, 2023

(54) ANTIBODY FRAGMENTS AND USES THEREOF FOR IMAGING CELLULAR ACTIVITY

(71) Applicant: Elephas Biosciences Corporation, Madison, WI (US)

(72) Inventors: Jonathan Daniel Oliner, Garrett Park, MD (US); Tomasz Zal, Madison, WI (US)

(21) Appl. No.: 18/118,977

(22) Filed: Mar. 8, 2023

Related U.S. Application Data
(60) Provisional application No. 63/317,681, filed on Mar. 8, 2022.

(51) Int. Cl. G01N 33/50; G01N 33/543

(52) U.S. Cl. CPC G01N 33/5082; G01N 33/5011; G01N 33/54346

(57) ABSTRACT

The present disclosure relates to materials and methods for live imaging of biological samples. In particular, the present invention provides antibody fragments for purposes of detecting (e.g., visualizing, monitoring, measuring, evaluating, and/or analyzing) cellular activity within biological samples (e.g., live tissue samples, live cell samples). In some aspects, the disclosure relates to use of antibody fragments (e.g., camelid antibody fragments) for detecting and measuring a response to an immunotherapy in live cells (e.g., live tumor fragments).

### RELATED APPLICATIONS

The present application claims priority to U.S. Provisional Application No. 63/317,681, filed Mar. 8, 2022, which is incorporated herein by reference in its entirety.

### FIELD OF THE INVENTION

The present disclosure relates to materials and methods for live imaging of biological samples. In particular, the present invention provides antibody fragments for purposes of detecting (e.g., visualizing, monitoring, measuring, evaluating, and/or analyzing) cellular activity within biological samples (e.g., live tissue samples, live cell samples). In some aspects, the disclosure relates to use of antibody fragments (e.g., camelid antibody fragments) for detecting and measuring a response to an immunotherapy in live tumor fragments.

### BACKGROUND

Immunotherapies have revolutionized the oncology landscape. However, monitoring and measuring patient responses to immunotherapy is difficult based solely on static correlates such as tumor infiltrating lymphocyte (TIL) localization and molecular signatures. Currently there is a lack of information about cell motility, proximity of cells to one another, or other dynamic behavior, in particular in response to drug treatments (e.g. immunotherapies). It is advantageous to measure the motility of specific cell subsets in tissue or other dynamic cellular behaviors of specific cell subsets in tissues because the motility is informative about drug activity in vivo. For example, drug may cause cessation of motility of a specific cell type or enhance cellular motility of a specific cell type in tissue. Therefore, it may be desirable to measure cellular motility or other dynamic behavior of specific cell types in tissues with and without drug treatment. However, many challenges exist for measuring motility of a specific cell type in tissue. For example, many reagents that bind to cells with a desirable specificity, namely antibodies, adversely interfere with cell motility or other cell behaviors. In addition, antibodies are slow to diffuse into tissues due to their large molecular weight. Accordingly, what is needed are improved methods for predicting patient responses to immunotherapy, including methods for measuring motility of one or more specific cell types, are needed.

The present invention addresses this need.

### SUMMARY

In some embodiments, provided herein is a method comprising contacting a living biological sample with one or more different sets of antibody fragments, and imaging cellular activity within the sample. In some embodiments, each set of antibody fragments comprises a different exogenous label. In some embodiments, each set of antibody fragments is specific for a specific cell type within the living biological sample. In some embodiments, imaging cellular activity within the sample comprises imaging cellular activity of the specific cell types within the living biological sample through detecting specific cell types engaged with exogenous labels associated with antibody fragments.

In some embodiments, imaging occurs over a period of time (e.g., 0.01 second, 0.05 seconds, 0.1 seconds, 0.5 seconds, 1 second, 10 seconds, 20 seconds, 1 minute, 1 hour, 1 day, etc.).

In some embodiments, the living biological sample is a living tissue sample. In some embodiments, the living biological sample is a living tumor fragment in culture. In some embodiments, the living biological sample is a mixture of different types of living cells.

In some embodiments, the cellular activity of specific cell types is related to immunological response. In some embodiments, the specific cell types include, but are not limited to, lymphocyte cells (e.g., T-cells, B-cells, natural killer (NK) cells), dendritic cells, neutrophils, and monocytes/macrophages. In some embodiments, the specific cell types comprise tumor infiltrating immune cells. For example, in some embodiments the tumor infiltrating immune cells comprise tumor infiltrating T-cells, tumor infiltrating B-cells, and tumor infiltrating NK cells. In some embodiments, the tumor infiltrating immune cells comprise T-cells (e.g. activated T-cells, CD8+ T-cells, CD4+ T cells) B-cells, and/or NK cells.

In some embodiments, the method further comprises quantifying the number of specific cell types in the living biological sample over a period of time. In some embodiments, the method further comprises measuring and/or monitoring the motility of the specific cell types within the living biological sample over a period of time. In some embodiments, the method further comprises monitoring the interaction between the specific cell types within the living biological sample over a period of time.

In some embodiments, the method further comprises analyzing the disease status of the living biological sample over a period of time through analyzing the interaction between the specific cell types within the living biological sample over the period of time. In some embodiments, the method further comprises analyzing an effect of an immunological challenge within the living biological sample over a period of time through analyzing the interaction between the specific cell types within the living biological sample over the period of time prior to, during, and after the immunological challenge. In some embodiments, the method further comprises analyzing an effect of a therapeutic agent within the living biological sample over a period of time through analyzing the interaction between the specific cell types within the living biological sample over the period of time prior to, during, and after contacting the living biological sample with the therapeutic agent. In some embodiments, the therapeutic agent is an immunotherapeutic agent. In some embodiments, the method further comprises analyzing immunological activity within the living biological sample over a period of time through analyzing the interaction between the specific cell types within the living biological sample.

In some embodiments, each of the one or more sets of antibody fragments comprises a specific Fab fragment. In some embodiments, the antibody fragment is a Fab or VHH or scFv fragment from a camelid antibody or a squalidae antibody or any antibody. The distinguishing features of the antibody fragments are the monovalent binding site. In some embodiments, antibody fragments have a molecular size that is less than 80 kdal. In some embodiments, antibody fragments have a molecular size that is less than 50 kdal.

In some embodiments, the exogenous label is a fluorescent label, and imaging the living biological sample comprises performing fluorescence imaging.

In some embodiments, the method further comprises one or more of visualizing, monitoring, measuring, evaluating, and analyzing cellular activity of the specific cell types within the living biological sample with software configured for one or more of visualizing, monitoring, measuring, evaluating, and analyzing cellular activity of the specific cell types within the living biological sample. In some embodiments, the method further comprises comparing the cellular activity of the specific cell types within the living biological sample with established norm controls for cellular responses (e.g., cellular activity consistent with healthy cellular activity, cellular activity consistent with abnormal cellular activity, cellular activity consistent with diseased cellular activity, etc.).

In some embodiments, the living biological sample is from a human subject. In some embodiments, the human subject has or is at risk of having cancer.

In some aspects, provided herein is a method comprising contacting a sample comprising live tumor fragments with at least one immunotherapeutic agent; and measuring a response to the at least one immunotherapeutic agent in the sample. In some embodiments, measuring a response to the at least one immunotherapeutic agent comprises evaluating at least one cell type in the sample. In some embodiments, the at least one cell type comprises a tumor infiltrating lymphocyte. In some embodiments, the tumor infiltrating lymphocyte comprises a T cell, a B cell, or an NK cell.

In some embodiments, the at least one cell type is bound to an antibody fragment comprising an exogenous label. In some embodiments, evaluating the at least one cell type comprises detecting the exogenous label, if present in the sample. For example, in some embodiments the exogenous label is detected by imaging the sample. In some embodiments, the exogenous label is a fluorescent label, and imaging the sample comprises performing fluorescence imaging. In some embodiments, the antibody fragment comprises a Fab fragment. In some embodiments, the antibody fragment is a Fab fragment from a camelid antibody or a squalidae antibody.

In some aspects, provided herein is a method comprising contacting a sample comprising live tumor fragments with at least one immunotherapeutic agent and an antibody fragment, wherein the antibody fragment comprises an exogenous label and binds to at least one cell type in the sample; and evaluating the at least one cell type in the sample. In some embodiments, evaluating the at least one cell type comprises detecting the exogenous label, if present in the sample. In some embodiments, the exogenous label is detected by imaging the sample. In some embodiments, the exogenous label is a fluorescent label, and imaging the sample comprises performing fluorescence imaging. In some embodiments, detection of the exogenous label indicates that the at least one cell type is present in the sample. In some embodiments, evaluating the at least one cell type comprises measuring motility of at least one cell.

In some embodiments, the at least one cell type comprises a tumor infiltrating lymphocyte. In some embodiments, the tumor infiltrating lymphocyte comprises a T cell, a B cell, or a natural killer (NK) cell. In some embodiments, the antibody fragment comprises a Fab fragment. For example, in some embodiments, the antibody fragment is a Fab fragment from a camelid antibody or a squalidae antibody.

In some aspects, provided herein are methods for evaluating a cellular response to an immunotherapeutic agent. In some embodiments, the method for evaluating a cellular response to an immunotherapeutic agent comprises contacting a sample comprising live tumor fragments with at least one immunotherapeutic agent and an antibody fragment, wherein the antibody fragment comprises an exogenous label and binds to at least one cell type in the sample, and measuring a response to the at least one immunotherapeutic agent in the sample by evaluating the at least one cell type in the sample. In some embodiments, evaluating the at least one cell type comprises detecting the exogenous label, if present in the sample. In some embodiments, the exogenous label is detected by imaging the sample. In some embodiments, the exogenous label is a fluorescent label, and imaging the sample comprises performing fluorescence imaging.

In some embodiments, the at least one cell type comprises a tumor infiltrating lymphocyte. In some embodiments, the tumor infiltrating lymphocyte comprises a T cell, a B cell, or a natural killer (NK) cell.

In some embodiments, the antibody fragment comprises a Fab fragment. In some embodiments, the antibody fragment is a Fab fragment from a camelid antibody or a squalidae antibody.

In some embodiments, evaluating the at least one cell type comprises measuring an amount of the at least one cell type in the sample and/or measuring the motility of the at least one cell type in the sample. In some embodiments, an increased amount of the at least one cell type and/or increased motility of the at least one cell type in the sample compared to the amount and/or motility of the at least one cell type in a control sample indicates a positive response to the at least one immunotherapeutic agent.

In some embodiments, provided herein is a method of evaluating a cellular response to an immunotherapeutic agent comprising contacting a sample comprising live tumor fragments with an antibody fragment, wherein the antibody fragment comprises an exogenous label and binds to at least one cell type in the sample; visualizing the sample to obtain a baseline measurement of the exogenous label contacting the sample with at least one immunotherapeutic agent and optionally contacting the sample a second time with the at least one antibody fragment comprising the exogenous label; visualizing the sample to obtain a second measurement of the exogenous label; and analyzing a difference between the baseline measurement and the second measurement to assess a cellular response to the immunotherapeutic agent. In some embodiments, provided herein is a method of evaluating a cellular response to an immunotherapeutic agent comprising contacting a sample with at least one immunotherapeutic agent and an antibody fragment, wherein the antibody fragment comprises an exogenous label and binds to at least one cell type in the sample; visualizing the sample to obtain a measurement of the exogenous label; contacting a control sample comprising live tumor fragments with the antibody fragment comprising the exogenous label; visualizing the control sample to obtain a control measurement of the exogenous label; and analyzing a difference between the measurement obtained in step b) and the measurement obtained in step d) to assess a cellular response to the immunotherapeutic agent.

In some embodiments, the exogenous label is a fluorescent label, and wherein visualizing comprises performing fluorescence imaging. In some embodiments, the at least one cell type comprises a tumor infiltrating lymphocyte. In some embodiments, the tumor infiltrating lymphocyte comprises a T cell, a B cell, or a natural killer (NK) cell. In some embodiments, the antibody fragment comprises a Fab fragment. For example, the antibody fragment may be a Fab fragment from a camelid antibody or a squalidae antibody.

For any of the methods described herein, the methods may further comprise measuring one or more biomarkers of immune activation in the sample. In some embodiments, the one or more biomarkers of immune activation are selected from interleukin-2 (Il-2), interleukin-4 (Il-4), interleukin-6 (Il-6), interleukin-10 (Il-10), interleukin-17A (Il-17A), Tumor necrosis factor alpha (TNF-α), soluble Fas (sFas), soluble Fas ligand (sFasL), interferon gamma (IFN-g), granzyme A, granzyme B, perforin, granulysin, interleukin-8 (I1-8), interferon gamma-induced protein 10 (IP-10), eotaxin, thymus and activation-regulated chemokine (TARC), monocyte chemoattractant protein-1 (MCP-1), RANTES, macrophage inflammatory protein (MIP)-1α, monokine induced by interferon-γ (MIG), epithelial-neutrophil activating peptide (ENA-78), MIP-3α, GROα, I-TAC, or MIP-1b. In some embodiments, the sample is obtained from a subject diagnosed with or at risk of having cancer.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques. In some embodiments, “about” may refer to variations of in some embodiments ±20%, in some embodiments ±10%, in some embodiments ±5%, in some embodiments ±1%, in some embodiments ±0.5%, and in some embodiments ±0.1% from the specified amount.

The phrase “and/or,” as used herein in the specification and in the claims, should be understood to mean “either or both” of the elements so conjoined, e.g., elements that are conjunctively present in some cases and disjunctively present in other cases.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” as used herein is any mammalian or non-mammalian subject. The subject may be a primate or a non-primate subject. In some embodiments, the subject is suspected of, at risk of, or diagnosed with cancer. In some embodiments, the subject is a human subject. The cancer can be any solid or hematologic malignancy. The cancer can be of any stage and/or grade. Non-limiting examples of cancer include cancers of head & neck, oral cavity, breast, ovary, uterus, gastro-intestinal, colorectal, pancreatic, prostate, brain and central nervous system, skin, thyroid, kidney, bladder, lung, liver, bone and other tissues.

“Tissue” or “tissue sample” as used herein is a biological material obtained from a subject. In some embodiments, the tissue contains or is suspected of containing tumor cells (also referred to as a tumor containing tissue). The terms tumor cells, cancerous cells, and malignant cells are used interchangeably herein. In some embodiments, the tissue is a solid tumor tissue. The tissue can be obtained from any organ or site in the body of the subject where a cancer has originated or where the cancer has metastasized to. A tissue can be obtained from a subject by any approach known to a person skilled in the art. The tissue can be obtained by surgical resection, surgical biopsy, investigational biopsy, bone marrow aspiration or any other therapeutic or diagnostic procedure performed on a subject suspected of or diagnosed with cancer.

“Tissue fragments” are fragments of the tissue sample that have detached from the tissue sample. In some embodiments where tissue fragments are obtained from a tumor containing tissue, the tissue fragments are referred to as “tumor fragments”. In some embodiments, tissue fragments are obtained by cutting the tissue in one or more dimensions. In some embodiments, the tissue fragments are obtained by cutting the tissue sample in all three dimensions, such as a first dimension, a second dimension, and a third dimension. In some embodiments, (such as in the case of a biopsy tissue sample) where the tissue sample already has the desired sizes in two dimensions, tissue fragments can be produced by cutting the tissue sample in only one dimension. The tissue fragments can be of various shapes, with non-limiting examples of shapes including cubes, square cuboids, rectangular cuboids, parallelogram prisms and the like. In some embodiments, the tissue fragments are substantially cubical in shape.

In some embodiments, the size of each tissue fragment is equal to or less than 1000 μm (such as 1000 μm, 500 μm, 450 μm, 400 μm, 350 μm, 300 μm, 250 μm, 200 μm, 100 μm or 50 μm) in at least one dimension. In some embodiments, the size of each tissue fragment is between 50 μm and 1000 μm in at least one dimension. In some embodiments, the size of each tissue fragment is between 100 μm and 500 μm in at least one dimension. In some embodiments, the size of each tissue fragment is between 150 μm and 350 μm in at least one dimension. In some embodiments, the size of each tissue fragment is between 50 μm and 500 μm (such as 50 μm, 100 μm, 150 μm, 200 pm, 250 μm, 300 μm, 350 μm, 400 μm, 450 μm or 500 μm) in at least two dimensions. In some embodiments, the size of each tissue fragment is between 100 μm and 350 μm in at least two dimensions. In some embodiments, the size of each tissue fragment is between 50 μm and 500 μm in all three dimensions. In some embodiments, the size of each tissue fragment is between 100 μm and 350 μm in all three dimensions. In some embodiments, each tissue fragment is between 300 μm and 350 μm in two dimensions and between 100 μm and 150 μm in a third dimension. In some embodiments, the tissue fragments are uniform in size. As used herein, uniform means substantially uniform, wherein the size of the tissue fragments are within ±30% of one another, in at least one dimension.

In some embodiments, the tissue fragments are live tissue fragments. Live tissue fragments obtained from a tumor containing tissue are referred to herein as “live tumor fragments” or “LTF”. Live tissue fragments (e.g. live tumor fragments) refer to fragments in which the viability of cells is not significantly altered compared to tissue that is freshly excised from the subject. In some embodiments, the tissue fragments are live tissue fragments wherein the cutting processes did not substantially reduce the number of viable cells that were present in the tissue sample. In some embodiments, the tissue fragments are live tissue fragments, such that one or more functional assays can be performed on the tissue fragments. In some embodiments, a live tissue or a live tissue fragment is one which has not been subjected to any tissue fixation techniques (such as formalin fixation). In some embodiments, cell viability may be determined by imaging techniques. In some embodiments, viable cells are cells with an intact cell membrane. According to some embodiments, the cell membranes of viable cells are largely impermeable to certain viability test molecules such propidium iodide, 7-AAD and the like.

As used herein, the term “antibody fragment” as used herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. In some embodiments, the antibody fragment is a monovalent antibody fragment. As used herein, the term “monovalent antibody fragment” refers to an antibody fragment that contains a singular antigen-binding site. Examples of suitable monovalent antibody fragments include, but are not limited to, VHH fragments, Fab fragments, Fab′ fragments, Fab′-SH fragments, Fv fragments, single-chain Fv (scFv) molecules, single-chain polypeptides containing only one light chain variable domain, single-chain polypeptides containing the three complementary determining regions (CDRs) of the light-chain variable domain, single-chain polypeptides containing only one heavy chain variable region, and single-chain polypeptides containing the three CDRs of the heavy chain variable region.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). For example, “treating cancer” may refer to reducing the size of a tumor, reducing the number of tumors, or completely eliminating tumors in a subject.

### DETAILED DESCRIPTION

In certain embodiments, the present invention provides antibody fragments for purposes of detecting (e.g., visualizing, monitoring, measuring, evaluating, and/or analyzing) cellular activity within a biological sample.

Such methods are not limited to detecting (e.g., visualizing, monitoring, measuring, evaluating, and/or analyzing) cellular activity within a specific type of a biological sample. In some embodiments, the biological sample is a tissue sample (e.g., live tissue sample). In some embodiments, the biological sample is a cell sample (e.g., live cell sample). In some embodiments, the cell sample is live tumor fragment culture. In some embodiments, the cell sample is a mixture of different types of living cells.

Such methods are not limited to detecting (e.g., visualizing, monitoring, measuring, evaluating, and/or analyzing) a particular type of cellular activity within a biological sample. In some embodiments, the cellular activity is related to an immunological response within the biological sample. For example, in some embodiments, the present invention provides methods for detecting (e.g., visualizing, monitoring, measuring, evaluating, and/or analyzing) an immune cell activity within a biological sample (e.g., live tumor fragment culture). In some embodiments, detecting cellular activity includes detecting the activity of various types of cells within a biological sample (e.g., motility of T cells, B cells, etc.).

Such methods are not limited to detecting (e.g., visualizing, monitoring, measuring, evaluating, and/or analyzing) a particular aspect of immune activity within a biological sample (e.g., living cell sample) (e.g., live tumor fragment culture). In some embodiments, the biological sample includes immune cells. The term “immune cell” as used herein refers to lymphocytes (T-cells, B-cells, natural killer cells), dendritic cells, neutrophils, and monocytes/macrophages. The term “immune cell” is inclusive of “tumor infiltrating” immune cells. The term “tumor infiltrating” refers to an immune cell that is located inside a tumor. The term “tumor infiltrating immune cell” is inclusive of “tumor infiltrating lymphocytes”, which refer to tumor infiltrating T-cells, B-cells, and natural killer (NK) cells. For example, the method may comprise evaluating T-cells (e.g. activated T-cells, CD8+ T-cells, CD4+ T cells) B-cells, and/or NK cells. In some embodiments, the methods involve detecting the activity (e.g., motility) of T-cells (e.g. activated T-cells, CD8+ T-cells, CD4+ T cells) B-cells, and/or NK cells within the biological sample.

In some embodiments, the method comprises quantifying the number (e.g., determining the amount) of cell types in the biological sample. For example, the number of T-cells (e.g. activated T-cells, CD8+ T-cells, CD4+ T cells) B-cells, and/or NK cells within a biological sample may be determined and quantified. In some embodiments, a response to an immunotherapeutic agent may be determined by measuring the amount of a cell type (e.g., T-cells (e.g. activated T-cells, CD8+ T-cells, CD4+ T cells) B-cells, and/or NK cells) in the sample. In some embodiments, evaluating at least one cell type comprises measuring motility of one or more cells of the given cell type in the sample. For example, evaluating a cell type may refer to measuring motility of tumor infiltrating lymphocytes in the sample. In some embodiments, the number of a given cell type and the motility of a given cell type are measured in the sample. In some embodiments, evaluating at least one cell type comprises evaluating proximity of certain cell types to other cell types. For example, evaluating at least one cell type may comprise evaluating proximity of tumor cells to immune cells.

In some embodiments, detecting (e.g., visualizing, monitoring, measuring, evaluating, and/or analyzing) various cell types within a biological sample is accomplished through labelling the various cell types. For example, in some embodiments a particular cell type is bound to an antibody fragment comprising an exogenous label. As such, evaluating the specific cell type may be performed by detecting the exogenous label.

In some embodiments, the method comprises contacting a biological sample (e.g. a sample comprising live tumor fragments) with one or more antibody fragments comprising exogenous labels specific for different cell types (e.g., immunological cell types) for purposes of detecting (e.g., visualizing, monitoring, measuring, evaluating, and/or analyzing) the activity of such different cell types. For example, in some embodiments, different antibody fragments comprising exogenous labels specific for T-cells (e.g. activated T-cells, CD8+ T-cells, CD4+ T cells), B-cells, and/or NK cells are provided. In some embodiments, upon exposure of the cell sample to such antibody fragments specific for various immune cell types (e.g., T-cells (e.g. activated T-cells, CD8+ T-cells, CD4+ T cells), B-cells, and/or NK cells) the antibody fragments bind to at least one cell type (e.g. tumor infiltrating immune cell) in the sample. In some embodiments, the method comprises evaluating the at least one cell type in the sample by detecting the exogenous label, if present in the sample.

In some embodiments, the antibody fragment comprises the antigen binding site of an intact antibody. In some embodiments, an antibody fragment is a portion of an intact antibody comprising the antigen-binding site or variable region. The portion does not include the constant heavy chain domains (e.g., CH2, CH3, or CH4, depending on the antibody isotype) of the Fc region of the intact antibody. In some embodiments, the antibody fragment is a monovalent antibody fragment. A “monovalent” antibody fragment refers to a fragment having only one antigen-binding site. Examples of suitable monovalent antibody fragments include, but are not limited to, VHH fragments, Fab fragments, Fab′ fragments, Fab′-SH fragments, Fv fragments, single-chain Fv (scFv) molecules, single-chain polypeptides containing only one light chain variable domain, single-chain polypeptides containing the three complementary determining regions (CDRs) of the light-chain variable domain, single-chain polypeptides containing only one heavy chain variable region, and single-chain polypeptides containing the three CDRs of the heavy chain variable region. For example, in some embodiments the antibody fragment comprises a Fab fragment.

As opposed to intact antibodies or bivalent or multivalent antibody fragments that contain a plurality of antigen-binding sites, monovalent fragments are advantageous because they provide improved tissue penetration and decreased interference with biological functions such as cellular motility. In some embodiments, monovalent fragments are also advantageous in that they may avoid inducing unwanted cell motility and/or cellular signaling events when binding to receptors compared to typical antibodies. For example, many reagents that bind to cell with a desirable specificity, namely antibodies, adversely interfere with cell motility or other cell behaviors. This problem may be caused by at least two properties of typical antibodies, namely bivalent or multivalent binding characteristics and Fc receptor binding. The bivalent or multivalent binding causes cross-linking of target ligands on cell surfaces which in turn triggers intracellular or extracellular biological signals that alter cell behavior. The Fc binding targets cells for Fc-mediated interactions with phagocytic cells often followed by cell engulfment and death as well as triggering multiple signaling pathways in either cell type. In addition, antibodies are slow to diffuse into tissues due to their large molecular weight. Taken together, whole antibodies are inadequate for the in-tissue cell behavior-based diagnostic purpose. Alternative methods include the genetic labeling of cell subsets with fluorescent proteins or non-specific dye labeling of purified cells. However, the genetic labeling method is not applicable to human-derived tissues. Likewise, the non-specific dye labeling method is inadequate because it requires cells to be removed from the tissue for the labeling followed by reintroducing the cells back into the tissue, which causes significant biological interference. Accordingly, the use of Fab fragments described herein possess many advantages over other methods in the art.

In some embodiments, the antibody fragment comprises a Fab fragment from a camelid antibody. Camelid antibodies are antibodies from the Camelidae family of mammals that include llamas, camels, and alpacas. These animals produce 2 main types of antibodies. One type of antibody camelids produce is the conventional antibody that is made up of 2 heavy chains and 2 light chains. The other type of camelid antibody is made up of only 2 heavy chains, and is also known as a heavy chain IgG (hcIgG). These antibodies do not contain the CH1 region, but they retain an antigen binding domain called the V_{H}H region. Accordingly, a Fab fragment from a camelid antibody is also referred to herein as a “V_{H}H” antibody, a “single domain antibody”, or a “nanobody”. In some embodiments, the antibody fragment comprises a Fab fragment from a shark (squalidae) antibody. A Fab fragment from a shark antibody is composed of a heavy chain homodimer retaining the antigen binding domain, and is also referred to herein as “VNAR”. In some embodiments, the antibody fragment (e.g. VHH antibody) is designed to bind to the at least one cell type of interest. For example, the antibody fragment may bind to a cell surface marker of the cell type of interest. For example, in some embodiments a VHH antibody containing an antigen binding domain that binds to a CD8+ T-cell may be used. As another example, in some embodiments a VHH antibody containing an antigen binding domain that binds to a CD4+ T-cell may be used. Any suitable cell surface marker may be the intended target of the antibody fragment (e.g. the VHH antibody), thus facilitating detection of any desired cell type within the sample.

In some embodiments, the antibody fragment further comprises an exogenous label. An exogenous label refers to a label that is added to (e.g., conjugated to) the antibody fragment. Any suitable type of exogenous label can be used, including an optical label (e.g., a fluorescent label), a magnetic label, an acoustic label and the like. In some embodiments, the exogenous label is a fluorescent label.

Such methods are not limited to a particular amount of time of detecting cellular activity within the biological sample. In some embodiments, the detecting occurs over any period of time (e.g., 0.01 second, 0.05 seconds, 0.1 seconds, 0.5 seconds, 1 second, 10 seconds, 20 seconds, 1 minute, 1 hour, 1 day, 1 month, 1 year, etc.).

In some embodiments, exposure of the biological sample to different antibody fragments specific for different cell types (e.g., immune cells) permits visualization of the different cell types within the biological sample (e.g., through detecting and monitoring motility of the labeled antibody fragments within the biological sample). In some embodiments, the visualization is real-time visualization thereby permitting visualization of the activity (e.g., motility) of the different cell types within the biological sample.

Such visualization may be used for any number of purposes. For example, in some embodiments, visualization of the immune cells (e.g., T-cells (e.g. activated T-cells, CD8+ T-cells, CD4+ T cells) B-cells, and/or NK cells) within the cell sample can be used to a observe a plurality of aspects of an immune response upon exposure to an immunological challenge (e.g., interaction and/or lack of interaction between different immune cell types before an immunological challenge, during an immunological challenge, and after an immunological challenge). In some embodiments, visualization of the immune cells within the cell sample can be used to a observe a plurality of aspects of an immune response upon exposure to a therapeutic agent (e.g., interaction and/or lack of interaction between different immune cell types before exposure to the therapeutic agent, during exposure to the therapeutic agent, and/or after exposure to the therapeutic agent).

In some embodiments, software is provided for detecting (e.g., visualizing, monitoring, measuring, evaluating, and/or analyzing) such a cellular response. In some embodiments, the software is configured to interpret the cellular activity (e.g., immune response). In some embodiments, the software is configured to compare cellular activity to established norm controls for various cellular responses (e.g., cellular behavior associated with a healthy response, abnormal response, etc.).

Imaging the sample thus facilitates knowledge of the presence and/or amount of detectable label, if present in the sample, which is indicative of the presence and/or amount of the cell type in the sample. In some embodiments, the exogenous label is a fluorescent label, and the methods comprise imaging the sample by fluorescence imaging. In some embodiments, imaging may be performed to determine the presence and/or amount of a given cell type in the sample, and/or to measure motility of a given cell type in the sample. For example, imaging may be performed to track motility of tumor infiltrating immune cells in the sample (e.g. using multiphoton microscopy for a desired period of time). Imaging may be performed for any suitable duration of time to determine motility of cells.

Such techniques further permit a monitoring and/or analyzing of an immune response over a period of time for purposes of monitoring/analyzing disease progression or regression, monitoring/analyzing the effect of a therapeutic intervention, etc.

In some embodiments, a single cell type is evaluated in the sample. For example, a first type of antibody fragment containing a first exogenous label (e.g. fluorescent label) may be added to the sample to bind to a first cell type. In some embodiments, multiple cell types are evaluated in the sample. In some embodiments, multiple cell types are evaluated sequentially. In other embodiments, multiple cell types are evaluated simultaneously. For example, a first type of antibody fragment containing a first exogenous label (e.g. a first fluorescent label) may be added to the sample to bind to a first cell type, and a second type of antibody fragment containing a second exogenous label (e.g. a second fluorescent label) that is different from the first exogenous label may be added to the sample to bind to a second cell type that is different from the first cell type. The first and second exogenous labels may be detected, thus facilitating evaluation of the two different cell types. In some embodiments, more than two types of labels are used, such that more than two cell types may be evaluated simultaneously. For example, in some embodiments three different types of antibody fragments comprising three different exogenous labels are used to evaluate three different cell types. In some embodiments, the first fluorescent label gives a first signal (e.g. green), the second fluorescent label gives a second signal (e.g. blue), the third fluorescent label gives a third signal (e.g. red), and so forth. Such methods may be advantageous for evaluating, for example, T-cells and B-cells in the sample, or multiple types of T-cells in the sample (e.g. CD8+ and CD4+ T-cells). Such methods may also be advantageous for evaluating, for example, the interactions between different types of immune cells. For example, the methods may comprise contacting the sample with multiple types of antibody fragments, each type comprising a different exogenous label. In some embodiments, a first antibody fragment comprising a first fluorescent label binds to a first cell type, a second antibody fragment comprising a second fluorescent label binds to a second cell type that is different from the first cell type, and so on. Such methods enable visualization of the exogenous label (e.g. the fluorescence signal) at time zero, and then monitoring the multiple fluorescent signals in the sample in response to an event, such as in response to addition of the immunotherapeutic agent. For example, the multiple fluorescent signals may be measured to monitor the amount of the various cell types in the sample, the motility of the various cell types in the sample, and/or the interactions between the various cell types in the sample.

In some embodiments, the methods described herein may be used to evaluate a cellular response to an immunotherapeutic agent. In some embodiments, provided herein is a method of evaluating a cellular response to an immunotherapeutic agent comprising contacting a sample comprising live tumor fragments with at least one immunotherapeutic agent and an antibody fragment. In some embodiments, the antibody fragment comprises an exogenous label and binds to at least one cell type in the sample. In some embodiments, the method further comprises measuring a response to the at least one immunotherapeutic agent in the sample by evaluating the at least one cell type in the sample. In some embodiments, evaluating the at least one cell type comprises detecting the exogenous label, if present in the sample. For example, the method may comprise imaging the sample to detect the exogenous label, if present in the sample. In some embodiments, the method comprises imaging the sample to determine the presence and/or amount of the exogenous label in the sample. In some embodiments, the method comprises imaging the sample to determine the motility of cells within the sample, such as by tracking the cells containing the exogenous label. As described above, in some embodiments more than one cell type may be evaluated in the sample, such as by using multiple types of antibody fragments, each type comprising a different exogenous label (e.g. fluorescent label). Evaluation of multiple cell types facilitates investigation of the amount, presence, and/or motility, of the multiple cell types, and/or interaction between the cell types present in the sample.

In some embodiments, the response to the therapeutic agent (e.g., immunotherapeutic agent) in the sample is compared to a control sample. The term “control sample” is used in the broadest sense and refers to multiple suitable controls. In some embodiments, the term “control sample” refers to a sample that is not contacted with the immunotherapeutic agent. For example, a “control sample” may be a sample that is contacted with an antibody fragment as described herein but is not contacted with an immunotherapeutic agent. In some embodiments the response to the immunotherapeutic agent (e.g. the presence/amount/motility of the at least one cell type) is compared to the same measurement (e.g. the presence/amount/motility of the at least one cell type) in the absence of the immunotherapeutic agent or following addition of a control agent. In some embodiments, an isotype control antibody serves as a control. In some embodiments, a first sample is used to evaluate response to the immunotherapeutic agent and a second sample is used to evaluate response to the control agent or response in the absence of the immunotherapeutic agent. The first and second sample may be obtained from the same subject and assessed in parallel. In such embodiments, the methods described herein may comprise contacting the control sample with at least one antibody fragment comprising an exogenous label, and visualizing the sample (e.g. imagine the sample) to obtain a control measurement of the exogenous signal in the sample without the addition of the immunotherapeutic agent. In some embodiments, the methods comprise contacting the control sample with multiple types of antibody fragments, each type of antibody fragment comprising a different exogenous label. For example, the methods may comprise contacting the control sample with a first antibody fragment comprising a first exogenous label (e.g. a first fluorescent label) and a second antibody fragment comprising a second exogenous label (e.g. a second fluorescent label) that is different from the first exogenous label. Such embodiments permit evaluation of multiple cell types in the control sample, such that a measurement of the presence and/or amount, motility, or interactions between multiple cell types can be evaluated.

In some embodiments, a “control sample” refers to the same sample in which the response to the immunotherapeutic agent is evaluated, wherein a baseline measurement is obtained in the sample prior to addition of the immunotherapeutic agent. In some embodiments, a baseline evaluation of the sample is performed (e.g. a baseline amount of a cell type is measured, a baseline motility of a cell type is measured, etc.) and a subsequent evaluation is conducted following contacting the sample with the immunotherapeutic agent. In some embodiments, a baseline evaluation of the sample is performed by contacting the sample with at least one antibody fragment comprising an exogenous label, and visualizing the sample (e.g. imaging the sample) to obtain a control measurement of the exogenous signal in the sample prior to the addition of the immunotherapeutic agent. In some embodiments, the methods comprise contacting the sample with multiple types of antibody fragments, each type of antibody fragment comprising a different exogenous label, such that a baseline measurement (e.g. a baseline amount, a baseline motility, a baseline level of interaction between two cell types, etc.) is obtained prior to contacting the sample with the immunotherapeutic agent. In such embodiments, the response following addition of the agent may be compared to the baseline evaluation.

In some embodiments, an increased amount of a given cell type in the sample following contact with the immunotherapeutic agent compared to a control/baseline indicates that the therapeutic agent has a positive response. A positive response indicates that the immunotherapeutic agent has a positive therapeutic effect, including increasing the number of cells having antitumor activity (e.g. tumor infiltrating lymphocytes) and/or increasing the motility of cells having antitumor activity. In some embodiments, a positive effect includes increased proximity of one cell type to another cell type. For example, in some embodiments a positive effect includes increased proximity of one cell type (e.g. effector T cells) to tumor cells. A positive response therefore also indicates that the immunotherapeutic agent may be a suitable cancer treatment for the subject from which the sample was obtained.

In some embodiments, the methods described herein further comprise measuring one or more biomarkers of immune activation in the sample. Suitable biomarkers of immune activation include, for example, interleukin-2 (Il-2), interleukin-4 (Il-4), interleukin-6 (Il-6), interleukin-10 (Il-10), interleukin-17A (Il-17A), Tumor necrosis factor alpha (TNF-α), soluble Fas (sFas), soluble Fas ligand (sFasL), interferon gamma (IFN-g), granzyme A, granzyme B, perforin, granulysin, interleukin-8 (Il-8), interferon gamma-induced protein 10 (IP-10), eotaxin, thymus and activation-regulated chemokine (TARC), monocyte chemoattractant protein-1 (MCP-1), RANTES, macrophage inflammatory protein (MIP)-1α, monokine induced by interferon-γ (MIG), epithelial-neutrophil activating peptide (ENA-78), MIP-3α, GROα, I-TAC, or MIP-1b. Any one or more biomarkers of immune activation may be measured in addition to evaluating the one or more cell types in the sample.

In some embodiments, the sample is obtained from a subject diagnosed with or at risk of having cancer. The cancer may be any type of cancer. In some embodiments, the subject is a human. The subject may be any age.

In some embodiments, the sample comprises live tumor fragments. Suitable methods for producing a sample comprising live tissue fragments (e.g. live tumor fragments) are described in U.S. patent application Ser. No. 17/566,154, the entire contents of which are incorporated herein by reference for all purposes. Generally speaking, live tumor fragments may be obtained by obtaining a tumor containing tissue sample from a subject, preserving/preparing the tissue as necessary for slicing, slicing the tissue into appropriate sizes under appropriate conditions to prevent a reduction in cell viability in the tissue, and maintaining the tissue under suitable conditions to maintain cell viability.

In some embodiments, the tissue, after being obtained from the subject, is first cut into tissue fragments. In some embodiments, the tissue fragments are placed in a suitable medium for extended preservation of cell viability, such as for transportation to a laboratory, where further processing of the tissue fragments takes place (such as sorting, imaging, culture etc.). In some embodiments, the tissue fragments are preserved under hypothermic preservation conditions. The term “hypothermic preservation” or “hypothermal preservation” mean preservation at a temperature below the physiological temperature (which is about 37° C.) but above the temperature of freezing, wherein biological processes are slowed down, thus allowing prolonged storage of a biological material. In some embodiments, hypothermic preservation is performed at temperatures between about 0° C. and about 10° C. A “hypothermally preserved tissue” or a “hypothermally preserved tissue fragment” refers to a tissue or a tissue fragment respectively, that has been preserved under hypothermic conditions. The terms “hypothermic preservation” and “cold preservation” have been used interchangeably. Similarly, the terms “hypothermic transport” and “cold transport” have been used interchangeably.

In some embodiments, the tissue fragments are preserved under cryopreservation conditions (such as at sub-zero temperature). The term “cryopreservation” means preservation of a biological material (such as tissue or tissue fragment) at a temperature below the freezing temperature (such as at sub-zero temperature). A “cryopreserved tissue” or a “cryopreserved tissue fragment” refers to a tissue or a tissue fragment respectively, that has been preserved at temperature below the freezing temperature (such as at sub-zero Celsius temperature). A sub-zero Celsius temperature (or sub-zero temperature) is any temperature below 0° C., such as less than about −10° C., less than about −20° C., less than about −50° C., less than about −100° C., less than about −120° C., less than about −150° C. and so on. In some embodiments, sub-zero temperature is a temperature of liquid nitrogen, such as the boiling temperature of liquid nitrogen at atmospheric pressure. In some embodiments, sub-zero temperature is a temperature between about 0° C. and about −200° C. In some embodiments, sub-zero temperature is a temperature of about −196° C.

In some embodiments, the tissue fragments are thawed for subsequent processing on reaching the destination site, such as a laboratory, where subsequent processing of the tissue fragments take place. In some embodiments, the tissue fragments are preserved under conditions, wherein after thawing, the viability of cells in the tissue fragments is not significantly reduced. In some embodiments, preservation of the tissue fragments under cryopreservation or hypothermic preservation conditions allows the tissue fragments to be stored for extended periods of time without significant reduction in cell viability or alterations in its metabolic profile. This allows great flexibility in the workflow and logistics. For example, it obviates any restriction of distance between the source site of tissue (such as a hospital) and the destination site (such as a laboratory) or of time elapsed between excision of the tissue and initiation of culture.

In some embodiments, the tissue is placed in a suitable medium for preservation before it is cut into tissue fragments. In some embodiments, the tissue is maintained under hypothermic preservation conditions in a suitable hypothermic preservation medium or under cryopreservation conditions in a suitable cryopreservation medium. The term “hypothermic preservation medium” means a preservation composition that would allow the biological material to withstand a temperature below the physiological temperature, such as a temperature below 10° C. to sustain its viability at such temperature. The terms “cryopreservation medium”, or “freezing medium”, refer to a medium in which a biological material is immersed before cryopreservation or freezing, or to medium which can be used to treat the biological material prior to freezing. A cryopreservation medium contains one or more cryoprotectants. In certain embodiments, a cryopreservation medium may be a freezing solution, a vitrification solution, and/or a mixture of such solutions. In certain embodiments, the cryopreservation medium refers to a medium for storing or freezing a biological material at a sub-zero Celsius temperature to sustain the viability of the tissue or the tissue fragments at that temperature. In some embodiments, the hypothermally preserved or the cryopreserved tissue is transported to a destination site, such as the laboratory for further processing. In some embodiments, the tissue is cut into tissue fragments after transportation. In some embodiments, a cryopreserved tissue is thawed before being cut into tissue fragments.

For any of the embodiments described herein, cutting the tissue can be performed manually, or it can be semi-automated or automated. Various suitable cutting devices may be employed for cutting the tissue. In some embodiments, the cutting device is configured to cut the tissue precisely and with minimal mechanical damage to the tissue or the tissue fragments. In non-limiting examples, cutting devices comprises a knife, a blade, a wire, a scalpel, a laser, and the like. In some embodiments, the cutting device comprises a plurality of blades. In some embodiments, the cutting device comprises a coated wire, such as a diamond particle coated steel wire (such as a diamond wire). In some embodiments, the cutting device comprises uniformly spaced wires (such as diamond wires or naked steel wired). In some embodiments, the cutting device comprises a cutting component. In some embodiments, the cutting component comprises at least one cutting member such as a knife, a blade, a wire, a scalpel, a laser, and the like. In some embodiments, the cutting device comprises three cutting components to cut the tissue in three dimensions, wherein each cutting component cuts the tissue in one dimension. The cutting device is configured to accurately and precisely cut a tissue into tissue fragments of a defined size. In some embodiments, the cutting device is configured to cut the tissue into tissue fragments based on a size input received from the user (user-defined). In some embodiments, the user-defined size input is based on physical properties of the tissue such as mechanical stiffness, frangibility and the like. In some embodiments, the cutting device is configured to cut the tissue into tissue fragments based on a pre-defined size input. In some embodiments, the cutting device is configured to cut the tissue into tissue fragments automatically and repeatedly until the entire tissue is cut into tissue fragments. In some embodiments, the cutting device is configured to cut the tissue into tissue fragments that are equal in size. As used herein, equal means substantially equal wherein the sizes of the tissue fragments are within ±20% of one another, in at least one dimension. In some embodiments, depending on the firmness of the tissue, the cutting device or components thereof are vibrated or rotated at user-defined or pre-defined frequency. The fragmentation settings of the cutting device such as thickness of tissue fragment, frequency, amplitude, speed etc. are user-defined or pre-defined.

In some embodiments, the tissue is cut under conditions of high oxygen concentration, that is an oxygen concentration greater than ambient oxygen concentration (such as greater than 21% or greater than 30% or greater than 50% or greater than 70%, or greater than 90% and the like). In some embodiments, the tissue is cut into tissue fragments in an oxygenated cutting medium.

In some embodiments, the tissue is prepared before cutting. For example, in some embodiments, the tissue is encapsulated in a gel matrix. A gel matrix can comprise a synthetic, a semi-synthetic or a natural component. In some embodiments, a gel matrix comprises at least one synthetic polymer or co-polymer, non-limiting examples of which includes poly(ethylene glycol) (PEG), poly(hydroxyethyl methacrylate) (PHEMA), poly(vinyl alcohol) (PVA), poly(acrylic acid) (PAA), poly(lactic acid), poly(caprolactone), poly(methycrylic acid) (PMMA), poly(lactic-co-glycolic acid) (PLGA), polyhydroxybutyric acid-valeric acid, poly(ethylene glycol)-diacrylate, poly(ethylene glycol)-vinyl sulfone and the like. In some embodiments, the polymers or co-polymers are further functionalized. In some embodiments, the tissue is contacted with a gel precursor. A gel precursor is a component that forms the gel matrix under suitable conditions of gelation. The gel precursor can be in any physical form such as in liquid or in solid form. In some embodiments, the gel matrix is formed by a covalent cross-linking of the gel precursors, while in some other embodiments the gel matrix is formed by a physical aggregation of the gel precursors. In some embodiments, depending upon the tissue type the percentages of the gel precursors and/or gelation conditions can be varied to obtain gel matrices of varying mechanical stiffness. In some embodiments, a gel matrix is formed when the gel precursor is irradiated with a light source. In some embodiments a gel matrix is formed when the gel precursor is subjected to a temperature change. While a skilled artisan can envisage multiple types of suitable gel matrices and gelation conditions, preferably the process of gelation to form the gel matrix should be fast and under conditions that cause minimal damage to the tissue or tissue fragments and that are inert to biological molecules. Further, the process of gelation and/or the gel matrix should not significantly alter the biological behavior of the cells in the tissue fragment. In some embodiments, gelation to form the gel matrix happens in less than 5 min (such as 4 min, 3 min, 2 min, 1 min or 30 second).

In some embodiments, the gel precursor is a PEG polymer such as a linear or a branched PEG polymer. A particularly suitable functionalized polymer can be, for example, a multi-arm, branched PEG polymer, such as a four-arm or an eight-arm PEG with terminal hydroxyl (—OH) groups that is functionalized with norbornene. In some embodiments, gelation to form the gel matrix happens in the presence of a suitable cross-linker such as a di-thiolated molecule (e.g., bi-functional PEG-dithiol). In some embodiments gel formation happens when the norbornene-functionalized multi-arm PEG polymer and bi-functional PEG-dithiol are irradiated with a light source.

In some embodiments, the tissue is contained within a sacrificial casing. While the gel matrix, the sacrificial casing, or both help to hold and stabilize the tissue during cutting, it is preferable not to have any trace of either during culture of the tissue fragments since residual gel matrix or residual sacrificial casing can interfere with nutrient availability, drug response and/or downstream analysis of the tissue fragments. In some embodiments, residual gel matrix, residual sacrificial casing or both are removed before the tissue fragments are contacted with the immunotherapeutic agent. In some embodiments, the step of cutting comprises driving the sacrificial casing containing the tissue towards the cutting component of the cutting device or driving the cutting component of the cutting device towards the sacrificial casing containing the tissue, wherein the cutting device cuts the tissue into tissue fragments by cutting through the sacrificial casing. In some embodiments, the sacrificial casing is formed of a material that can be cut with a cutting mechanism. Non-limiting examples of materials of the sacrificial casing include polypropylene, wax, silicone (such as Polydimethylsiloxane (PDMS)) and various thermoplastic elastomers. The material should preferably be biocompatible and non-toxic to avoid damaging or altering the tissue properties. In some embodiments, the sacrificial casing comprises a hollow cavity to house the tissue within. In some embodiments, the sacrificial casing comprises a groove to hold the tissue.

In some embodiments, the tissue fragments (e.g. live tumor fragments) are maintained in suitable culture conditions within a culture platform. A culture platform is any suitable culture device or system for culturing tissue fragments. Non-limiting examples of a culture platform include a well-plate or a fluidic device. In some embodiments, the culture platform comprises an oxygen-permeable material. Various types of oxygen-permeable materials may be employed. In some embodiments, the oxygen-permeable material comprises a fluoropolymer, non-limiting examples of which include FEP (fluorinated ethylene-propylene), TFE (tetrafluoroethylene), PFA (perfluoroalkoxy), PVF (polyvinylfluoride), PVDF (polyvinylidene fluoride), PTFE (polytetrafluoroethylene), PCTFE (polychlorotrifluoroethylene), ETFE (polyethylenetetrafluoroethylene), ECTFE (polyethylenechlorotrifluoroethylene), FFPM/FFKM (perfluoroelastomer), FPM/FKM (chlorotrifluoroethylenevinylidene fluoride), PFPE (perfluoropolyether), MFA (tetrafuoroethylene and perfuoromethyl vinyl-ether copolymer), CTFE/VDF (chlorotrifuoroethylene-vinylidene fluoride copolymer), and TFE/HFP (tetrafuoroethylene-hexafuoropropylene copolymer), or mixtures thereof. In some embodiments, the oxygen-permeable material comprises cyclic olefin polymer (COP) and cyclic olefin copolymers (COC). In some embodiments, the oxygen-permeable material comprises a silicone material (e.g., polydimethylsiloxane (PDMS)). In some embodiments, the culture platform is formed of extremely thin sections of one or more oxygen-permeable material. In some embodiments, regions of culture platform include chambers of the culture platform. Chambers of the culture platform can be wells of a well-plate or channels of a fluidic device. In some embodiments, the culture platform is configured for perfusion culture. In some embodiments, the culture platform is configured for non-perfused, static culture. In some embodiments, the culture platform is formed of a material that is optically transparent, thereby allowing optical investigation of the tissue fragments while the tissue fragments are within the chambers of the culture platform.

In some embodiments, the method comprises contacting a sample with at least one immunotherapeutic agent. The immunotherapeutic agent may be any immunotherapeutic agent with the potential for treatment of cancer. The immunotherapeutic agent may comprise an immune checkpoint inhibitor, a monoclonal antibody, a cancer treatment vaccine, or an immune system modulators. For example, the immunotherapeutic agent may be an immune checkpoint inhibitor targeting PD-1 and/or PD-L1, CTLA-4, or other immune checkpoint proteins. For example, the immunotherapeutic agent may be an immune checkpoint inhibitor selected from nivolumab, pembrolizumab, ipilimumab, atezolizumab, avelumab, and durvalumab. As another example, the immune checkpoint inhibitor may be a monoclonal antibody (e.g. atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obitunuzumab, ofatumumab, oralatumab, panitumumab, pembrolizumab, pertuzumab, ramucirumab, rituximab, trastuzumab, gemtuzumab ozogamicin, bentruximab vedotin, trastuzumab emtansine, inotuzumab ozogamicin, polatuzumab vedotin, efortumab vedotin, trastuzumab deruxtecan, Sacituzumab govitecan, moxetumuomab pasudotox, ibritumomab tiuxetan, iodine tositumuomab, blatinumomab). Any immunotherapeutic agent may be used, and the methods described herein may be used to evaluate the therapeutic potential of the agent for a given subject from which a sample was obtained.

### EXAMPLES

The following examples are intended only to illustrate methods and embodiments in accordance with the invention, and as such should not be construed as imposing limitations upon the claims.

### Example 1

Background: Immunotherapies have revolutionized the oncology landscape. However, predicting patient responses to immunotherapy is difficult based solely on static correlates such as tumor infiltrating lymphocyte (TIL) localization and molecular signatures. Accordingly, what is needed are improved methods for predicting patient responses to immunotherapy.

Overview: Anti-tumor immune response depends on motile surveillance by tumor infiltrating lymphocytes (TIL), which recognize antigenic determinants and engage target cells in serial stop-and-go interactions that result in cell killing. However, the hostile tumor microenvironment can cause TIL dysfunction and lack of cytotoxicity, which may be manifested as either suppressed or aimless TIL motility. Described herein is a diagnostic platform using live tumor fragments (LTF) that preserves the tumor microenvironment and its immune cells, thus enabling determination of which immunotherapy works best for a given patient.

Methods and Results: Antibody-based labeling of live tissues is hampered by slow diffusion and function-altering cross-linking. To overcome these limitations, small camelid-derived monovalent antibodies (nanobodies) were used to monitor TIL motility in LTFs.

Human tumor excisions were obtained from the Univ. of Wisconsin (IRB approved), and CT26 tumors were grown in mice subcutaneously. Live tumors were cut into 300×300 μm fragments of 100-300 μm thickness, sorted into multi-well plates, and cultured for 48 or 72 h in the presence or absence of antiPDl (nivolumab or mouse equivalent), anti-PD1 plus anti-CTLA4 (ipilimumab or mouse equivalent), or concanavalin A (ConA) as a positive control.

Immune responsiveness of LTFs to immunotherapy was ascertained by flow cytometry and secreted protein assays. CD8+ cells in LTFs were labeled using an anti-hCD8a camelid VHH nanobody covalently labeled with AF594. The same reagent or a mouse anti-hCD8a whole IgG antibody was used to stain human peripheral blood mononuclear cells, and staining patterns were compared by flow cytometry. Multiphoton microscopy revealed LTF collagen fibrils and cellular autofluorescence. The fluorescent anti-CD8a nanobody, but not a similarly labeled whole IgG, yielded good contrast and fast staining of two cell subsets. The smaller cells were 12 μm in diameter, cell surface-stained, and lacking autofluorescence, consistent with T cells. The larger cells were elongated, ramified, intracellularly stained, and distinctly autofluorescent, consistent with macrophages.

The motility of CD8+ cells was tracked using 3D multiphoton microscopy for 30 min. Using multiphoton microscopy and CD8-binding nanobodies, vigorous CD8+ T cell motility was observed in human LTFs, with a speed of 10 μm/min along collagenous structures. In contrast, larger cells exhibited only slow motility. These results show that the motility of human CD8+ T lymphocytes can be revealed in LTF culture using a fluorescent CD8-binding camelid nanobody, likely due to its small size and monovalent binding. The autofluorescence of larger, immotile cells was consistent with tumor-associated macrophages. Based on this distinction, T cells could be distinguished from the macrophages clearly. These results support the use of camelid-derived VHH and other small monovalent reagents for live tissue lymphocyte tracking, such as in evaluation of TIL response to immunotherapy in an LTF assay.

Supernatant cytokines were measured using bead immunoassay and T cell markers by flow cytometry. T cells were retained within LTFs, and the proportion of lymphocytes in LTFs was independent of fragment thickness (1.6%/2.1% for 300 pm, 1.4%/2.3% for 200 μm and 1.4%/2.8% for 100 μm thickness, for CD4+ and CD8+ T cells, respectively). Total cell viability and T cell viability exceeded 80% at 48 h, and 3D LTF structure remained intact for at least 48 h. LTFs were treated with anti-PD1, anti-PD1 plus anti-CTLA4, or ConA and confirmed the presence of IFN-γ and 10 (mouse) or 16 (human) other cytokines associated with immune activation in both the ConA and anti-PD1 treated samples, but not the control. In human LTFs, cytokine panel upregulation was observed for anti-PD1 vs. control (p=1.1e-5) and anti-PD1 plus anti-CTLA4 vs. anti-PD1 (p=3.7e-9).

In summary, provided herein is an LTF platform having an immuno-competent tumor microenvironment which allows for detection of cellular and secreted immune response markers, comparison of alternative treatments, and tracking the surveillance activity of infiltrating T cells.

1. A method comprising
contacting a living biological sample with one or more sets of antibody fragments, wherein each set of antibody fragments comprises a different exogenous label, wherein each set of antibody fragments is specific for a specific cell type within the living biological sample, wherein each of the one or more sets of antibody fragments comprises a specific Fab fragment or a specific scFv fragment, and
imaging cellular activity of the specific cell types within the living biological sample through detecting specific cell types engaged with exogenous labels associated with antibody fragments, wherein the imaging occurs over a period of time (e.g., 0.01 second, 0.05 seconds, 0.1 seconds, 0.5 seconds, 1 second, 10 seconds, 20 seconds, 1 minute, 1 hour, 1 day, etc.).

2. (canceled)

3. The method of claim 1,
wherein the living biological sample is a living tissue sample, and/or
wherein the living biological sample is a living tumor fragment culture, and/or
wherein the living biological sample is a mixture of different types of living cells.

4-5. (canceled)

6. The method of claim 1, the cellular activity of specific cell types is related to immunological response.

7. The method of claim 6, wherein the specific cell types include, but are not limited to, lymphocyte cells (e.g., T-cells, B-cells, natural killer (NK) cells), dendritic cells, neutrophils, and monocytes/macrophages.

8. The method of claim 6, wherein the specific cell types comprise tumor infiltrating immune cells, wherein the tumor infiltrating immune cells comprise one or more of tumor infiltrating T-cells, tumor infiltrating B-cells, tumor infiltrating NK cells, T-cells (e.g. activated T-cells, CD8+ T-cells, CD4+ T cells), B-cells, and NK cells.

9-10. (canceled)

11. The method of claim 1, further comprising one or more of:
quantifying the number of specific cell types in the living biological sample over a period of time,
measuring and/or monitoring the motility of the specific cell types within the living biological sample over a period of time,
monitoring the interaction between the specific cell types within the living biological sample over a period of time,
analyzing the disease status of the living biological sample over a period of time through analyzing the interaction between the specific cell types within the living biological sample over the period of time,
analyzing an effect of an immunological challenge within the living biological sample over a period of time through analyzing the interaction between the specific cell types within the living biological sample over the period of time prior to, during, and after the immunological challenge,
analyzing an effect of a therapeutic agent (e.g., immunotherapeutic agent) within the living biological sample over a period of time through analyzing the interaction between the specific cell types within the living biological sample over the period of time prior to, during, and after contacting the living biological sample with the therapeutic agent, and
analyzing immunological activity within the living biological sample over a period of time through analyzing the interaction between the specific cell types within the living biological sample.

12-19. (canceled)

20. The method of claim 1, wherein the antibody fragment is a Fab fragment from a camelid antibody or a squalidae antibody.

21. The method of claim 1, wherein each of the exogenous labels is a fluorescent label, and wherein imaging the living biological sample comprises performing fluorescence imaging.

22. The method of claim 1, further comprising
one or more of visualizing, monitoring, measuring, evaluating, and analyzing cellular activity of the specific cell types within the living biological sample with software configured for one or more of visualizing, monitoring, measuring, evaluating, and analyzing cellular activity of the specific cell types within the living biological sample, and/or
comparing the cellular activity of the specific cell types within the living biological sample with established norm controls for cellular responses (e.g., cellular activity consistent with healthy cellular activity, cellular activity abnormal cellular activity, cellular activity consistent with diseased cellular activity, etc.).

23. (canceled)

24. The method of claim 1, wherein the living biological sample is from a human subject who has or is at risk of having cancer.

25-52. (canceled)

53. A method of evaluating a cellular response to an immunotherapeutic agent, the method comprising:
contacting a sample comprising live tumor fragments an antibody fragment, wherein the antibody fragment comprises an exogenous label and binds to at least one cell type in the sample, wherein the antibody fragment is a Fab fragment from a camelid antibody or a squalidae antibody;
visualizing the sample to obtain a baseline measurement of the exogenous label;
contacting the sample with at least one immunotherapeutic agent and optionally contacting the sample a second time with the at least one antibody fragment comprising the exogenous label;
visualizing the sample to obtain a second measurement of the exogenous label; and
analyzing a difference between the baseline measurement and the second measurement to assess a cellular response to the immunotherapeutic agent.

54. A method of evaluating a cellular response to an immunotherapeutic agent, the method comprising:
a) contacting a sample with at least one immunotherapeutic agent and an antibody fragment, wherein the antibody fragment comprises an exogenous label and binds to at least one cell type in the sample, wherein the antibody fragment is a Fab fragment from a camelid antibody or a squalidac antibody;
b) visualizing the sample to obtain a measurement of the exogenous label;
c) contacting a control sample comprising live tumor fragments with the antibody fragment comprising the exogenous label;
d) visualizing the control sample to obtain a control measurement of the exogenous label; and
e) analyzing a difference between the measurement obtained in step b) and the measurement of obtained in step d) to assess a cellular response to the immunotherapeutic agent.

55. The method of claim 53, wherein the exogenous label is a fluorescent label, and wherein visualizing comprises performing fluorescence imaging.

56. The method any one of claim 53, wherein the at least one cell type comprises a tumor infiltrating lymphocyte, wherein the tumor infiltrating lymphocyte comprises a T cell, a B cell, or a natural killer (NK) cell.

58-61. (canceled)

62. The method of any one of claim 53, wherein the sample is obtained from a subject diagnosed with or at risk of having cancer.

63. The method of claim 54, wherein the exogenous label is a fluorescent label, and wherein visualizing comprises performing fluorescence imaging.

64. The method any one of claim 54, wherein the at least one cell type comprises a tumor infiltrating lymphocyte, wherein the tumor infiltrating lymphocyte comprises a T cell, a B cell, or a natural killer (NK) cell.

65. The method of any one of claim 54, wherein the sample is obtained from a subject diagnosed with or at risk of having cancer.
